(19) United States
(12) Patent Application Publication
(10) Pub. No.: US 2008/0027450 A1
(43) Pub. Date: Jan. 31, 2008

(54) Lateral Edge Marker

(76) Inventor: Douglas Raymond, Randolph, MA (US)

Correspondence Address: PHILIP S. JOHNSON;JOHNSON & JOHNSON, ONE JOHNSON & JOHNSON PLAZA, NEW BRUNSWICK, NJ, 08933-7003, US

(21) Appl. No.: 11/460,811

(22) Filed: Jul. 28, 2006

(51) Int. Cl. A61F 5/00

(52) U.S. Cl. 606/86

(57) ABSTRACT

A device that provides lateral markers for the surgeon as an indication of the permissible outer boundary limits of insertion of an intervertebral motion disc into the disc space.

### BACKGROUND OF THE INVENTION

Typically, a problematic intervertebral disc has been treated by removing the disc and fusing the adjacent vertebrae. In some conventional anterior cervical disc fusion procedures, distraction pins are placed at the approximate midline of the adjacent vertebrae and are used as a means of distraction. In a fusion procedure, midline placement of these pins is less important than providing full coverage of the vertebral endplates because physiological motion is not being restored. The outcome of the fusion is less dependent upon the correct identification of the midline and more dependent upon the promotion of fusion.

Recently, there have been attempts to manage disc degeneration by removing the problematic disc and replacing it with an articulating intervertebral motion disc. The placement of a motion disc requires a change in thinking and a shifting of goals that were once standard practice for inserting a fusion device.

Proper placement of a motion disc prosthesis is critically important to both restoration of physiologic motion and reduction of complications risks. In a motion disc implantation procedure, one of the prime end goals of a surgeon is to maximize coverage of the adjacent vertebral body endplates by the motion disc. To do this effectively, the surgeon must know the location of the prosthesis endplates during implantation. One conventional technique for insuring proper location of the prosthesis during implantation is to identify the midlines of the vertebral bodies adjacent the operable disc space, and to key instrument and implant insertion into the disc space off of those midlines.

Surgeons currently identify the midline of a vertebral body by locating the spinous processes of the vertebrae using an anterior-posterior fluoroscopic image. A marking pin is then placed on the anterior face of the vertebral body such that it is in midway between the edges of the spinous processes. In placing the marking pin, the surgeon assumes that the line created from the tips of each spinous process is equidistant from both the left and right lateral edges of the operable vertebral body. However, due to variations in anatomy, this assumption is often an inaccurate one and so there is a risk of malpositioning the motion device to a location lateral to the actual midline. Moreover, even in the case of no anatomical variation, keying off of the spinous processes to obtain the midline only results in an average midline, resulting from the average spinous process locations seen on the A-P fluoroscopy image, not the segmental midline of the pertinent functional spinal unit. Lastly, even when the midline marker is placed correctly, it is often visually obscured during the surgery, and so becomes of limited use.

Some conventional instruments include a unit that fixes to the anterior surface of the operable level. These instruments provide windows through which instruments are passed to perform the surgery. These devices are cumbersome, greatly restrict the surgeon's visualization, require overdistraction, and reduce the surgeon's workable space.

### SUMMARY OF THE INVENTION

The present invention relates to a device that provides lateral markers for the surgeon as an indication of the permissible outer boundary limits of insertion of an intervertebral motion disc into the disc space. The present invention is designed to provide the surgeon with a visually determined working space that defines the outer boundary limits of placement of a motion disc prosthesis in a disc space. By using the device of the present invention, the midline of the operable motion segment is also achieved and therefore maximum endplate coverage is obtained. As this device can be inserted in the early stages of the implant procedure, it can also be used to define outer boundary limits for other steps in the implantation procedure, such as endplate preparation (e.g., rasping) and trialing. This lateral edge marker device should also reduce the surgeon's reliance upon fluoroscopy, which is used to ensure the prosthesis is located upon the midline and that it has properly maximized endplate coverage.

Therefore, in accordance with the present invention, there is provided a lateral edge marker for guiding the insertion of an intervertebral motion disc, comprising:
a) a bone anchor having a distal end portion adapted for fixation to a vertebral body, a proximal end and a longitudinal shaft therebetween, and
b) a pair of arms extending transversely from the bone anchor, each arm having a lateral edge.

Also in accordance with the present invention, there is provided a method of inserting an intervertebral motion disc, comprising the steps of:
a) providing an upper and lower lateral edge marker, each marker comprising:
   i) a bone anchor having a distal end portion adapted for fixation to a vertebral body, a proximal end and a longitudinal shaft therebetween,
   and
   ii) a pair of arms extending from the transversely from the bone anchor, each arm having a lateral edge
b) inserting the upper lateral edge marker into an upper vertebra,
c) inserting the lower lateral edge marker into a lower vertebra, so that the four edges define a window, and
d) inserting the motion disc through the window and into a disc space between the upper and lower vertebrae.

The lateral edge marker of the present invention can be used to both mark the midline as well as define the outer boundary lateral edges of the vertebral body. In contrast, currently available midline marking pins give no indication as to the permissible lateral edge location of the prosthesis. Currently available distraction pins are also able to mark the midline, but also fall short in providing any indication of the permissible lateral edge location.

The lateral edge markers of the present invention can be used during endplate preparation to ensure that the surgeon properly prepares only the desired region of the vertebral endplates. Currently available endplate preparation tools and trialing devices are marked such that they align with the midline marker or distraction pins, and a surgeon tries to prepare the endplates with these conventional instruments by visually lining up this etch line with the midline marker. However, this process can often result in lateral misplacement of the targeted endplate by as much as 2-3 mm. The lateral edge marker of the present invention may reduce this undesired result by providing a visual indication of the amount of permissible lateral drift allowed in endplate preparation, while simultaneously improving the surgeon's ability to stay within their predetermined space.

Also, as the device of the present invention provides both right and left visual boundaries, the surgeon now has two lateral indicators to use as a visual guide instead of just a single midline.

In contrast to the bulky windows provided by some conventional units, the lateral edge marker of the present invention similarly defines a workable space, but does so by providing visual indicators and so eliminates the risks inherent in the conventional devices.

### DESCRIPTION OF THE DRAWINGS

FIG. 1 is a perspective view of a lateral edge marker of the present invention.

FIGS. 2a and 2b are top views of lateral edge markers having curved arms.

FIG. 2c is a top view of a lateral edge marker having parallel edges.

FIG. 3 is a perspective view of a modular lateral edge marker.

FIG. 4 is a posterior view of a pair of lateral edge markers of the present invention inserted into opposing adjacent vertebrae.

FIGS. 5a-5c are perspective, side and bottom views of an insertion instrument adapted to insert the lateral edge marker.

FIG. 6 is a perspective view of a lateral edge marker held by the insertion instrument of FIG. 5a.

FIGS. 7a-7b are exploded and assembled views of a modular lateral edge marker incorporating a Caspar Pin.

### DETAILED DESCRIPTION OF THE INVENTION

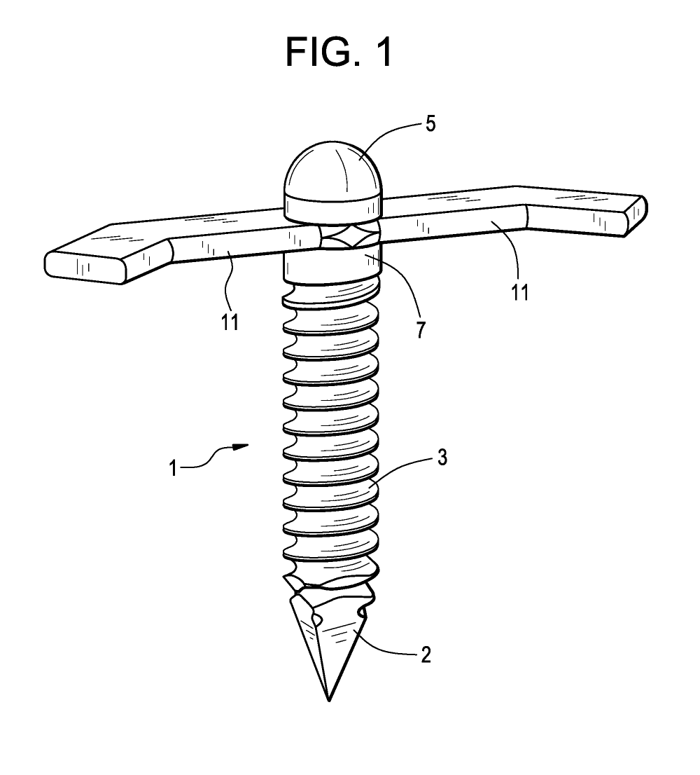

Now referring to FIG. 1, in some embodiments, the bone anchor 1 is a temporary fixation pin that is attached to an anterior surface of the vertebral body. The pin typically has a distal end portion 2, an intermediate portion 7 comprising a threaded shaft 3 adapted for bony fixation, and a proximal end 5. So that endplate coverage is maximized, preferably, the bone anchor of each marker is inserted into a midline of its respective vertebra.

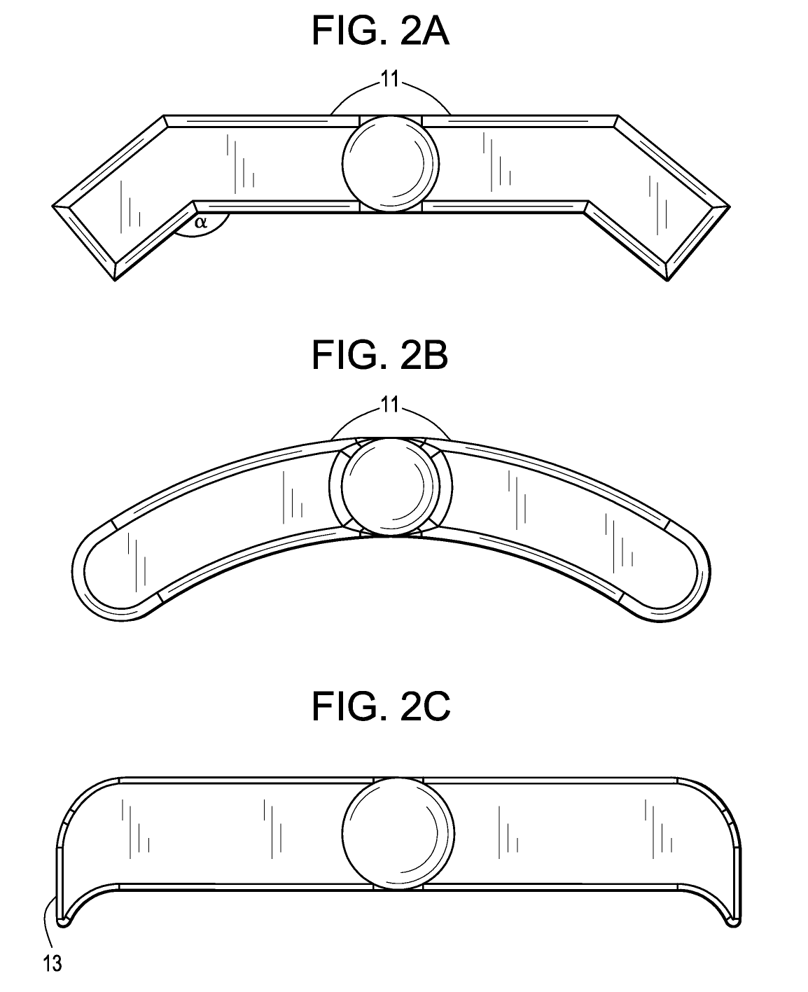

Extending from the intermediate portion of the temporary fixation pin is a pair of lateral arms 11 or wings. These arms are dimensioned to define the outer boundaries of the disc prosthesis and/or the lateral edges of the disc space. Now referring to FIGS. 2a and 2b, in preferred embodiments, the arms 11 form an angle α of between about 90 and 180 degrees. Providing the arms to form an angle α of between about 90 and less than 180 degrees preferably allows the surgeon to place the anchor safely into the anterior face of the vertebra, while allowing each arm to extend down towards the vertebral endplate, thereby allowing the lateral edge of each arm to be adjacent the vertebral endplate and shortening the distance between the opposing edges and easing the task of visualizing the window.

Now referring to FIG. 2c, in preferred embodiments, each arm has an outer lateral edge 13, and the lateral edges are substantially parallel to each other. When a pair of such markers are inserted into upper and lower vertebral bodies, they may be oriented so that the lateral edges of the upper and lower markers are substantially parallel to each other, and the edges should run directly towards the opposing edges on the opposing marker, thereby making easier the task of visualizing the window of permissible entry.

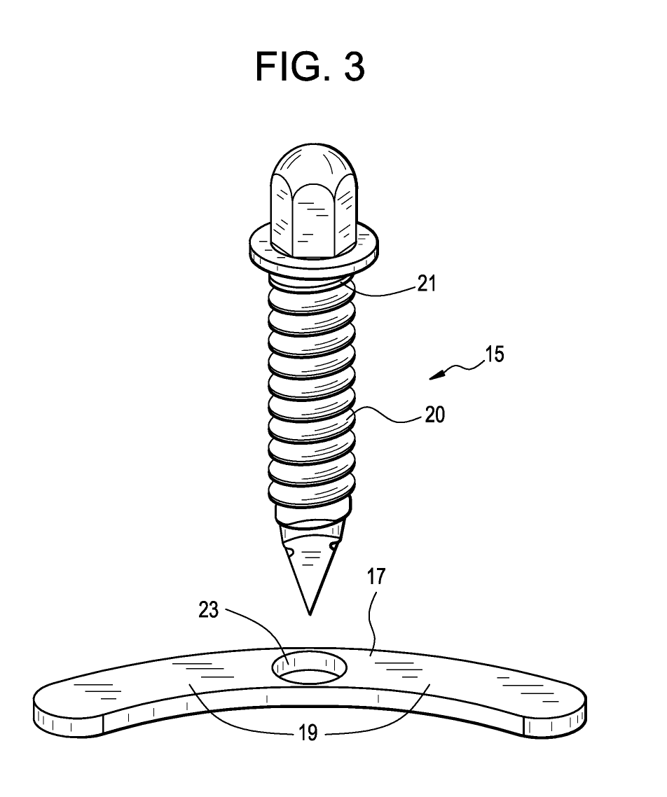

Now referring to FIG. 3, in one embodiment, the marker is a two piece design, and includes a standard distraction pin 15 having a shaft 20 and a modular ring 17 having a pair of arms 19 extending therefrom. The distraction pin can have an engagement surface 21 adapted to engage and hold the inner surface 23 of the modular ring. In use, the distraction pin is inserted into a vertebral body and the modular ring is inserted down and friction fit upon the shaft of the distraction pin. The ring can also be be locked onto the shaft via an anti-rotation feature such as a nub.

Therefore, in accordance with the present invention, there is provided a modular lateral edge marker, comprising
a) a standard distraction pin having a shaft, and
b) a ring having a pair of arms extending therefrom,
   wherein the ring is adapted to be received upon the shaft of the distraction pin.

The lateral edge marker is sized such that it can accommodate all sizes of disc prostheses and/or vertebral body widths. The lateral arms can be sized to span a variety of overall distances to accommodate all sizes of disc prosthesis and vertebral body widths.

In some embodiments, the arms or wings of the lateral edge marker are made of a material that is flexible. This flexibility enables the surgeon intraoperatively modify the arm edge to arm edge dimension and accommodate all desired widths.

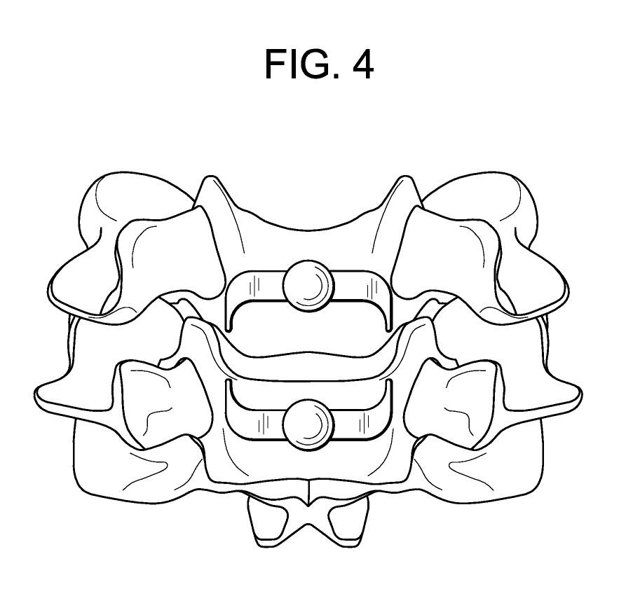

Now referring to FIG. 4, a pair of the lateral edge markers of the present invention are used in tandem by placing the bone anchor portion of each marker directly upon the midline of the respective upper and lower vertebral bodies.

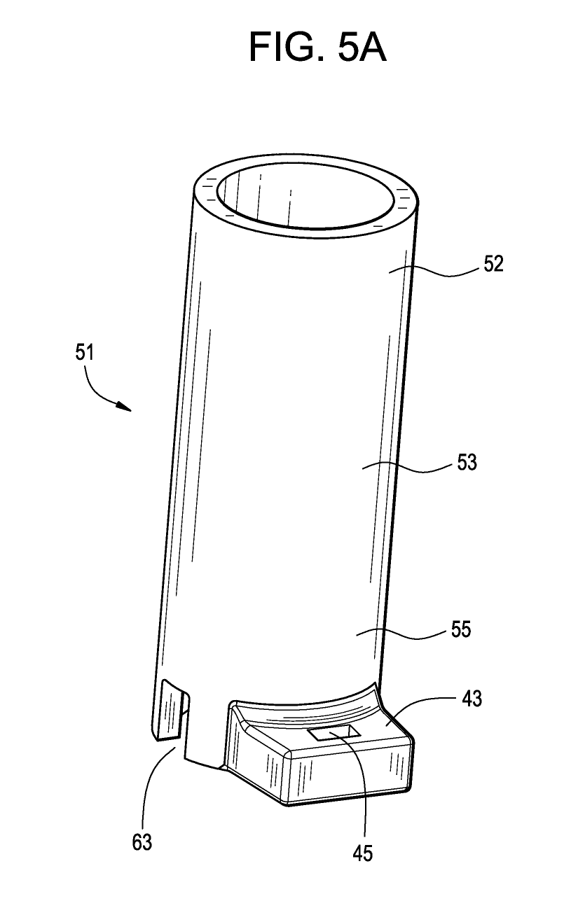

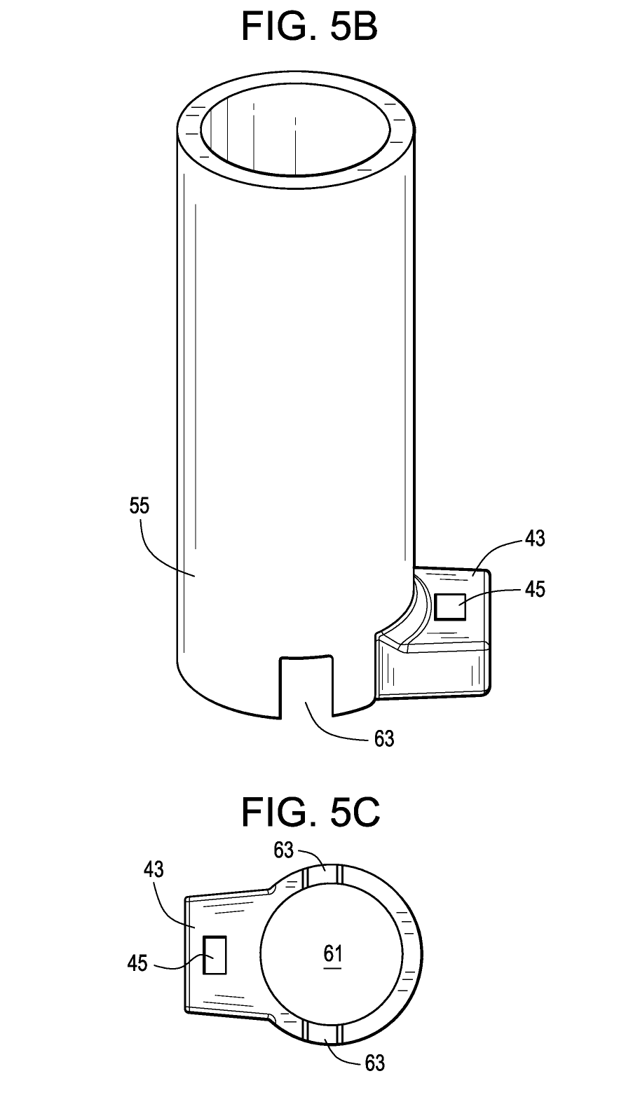

Now referring to FIGS. 5a-5c, the lateral edge marker of the present invention is preferably placed into a vertebral body with the help of insertion tool 51. The insertion tool has a proximal handle 52, an intermediate shaft 53, and a distal end portion 55. The distal end portion of the tool has a flange 43 extending laterally therefrom and a rectangular throughhole 45 passing axially through the flange. In use, this axial throughhole is passed over a midline marker that has been placed upon the midline in the intervertebral disc, thereby centering the insertion tool upon the midline.

The distal end portion also has an axial recess that comprises a central deep recess 61 for accepting the proximal portion of the bone anchor and a pair of more shallow lateral recessed portions 63 for accepting an inner portion of each arm.

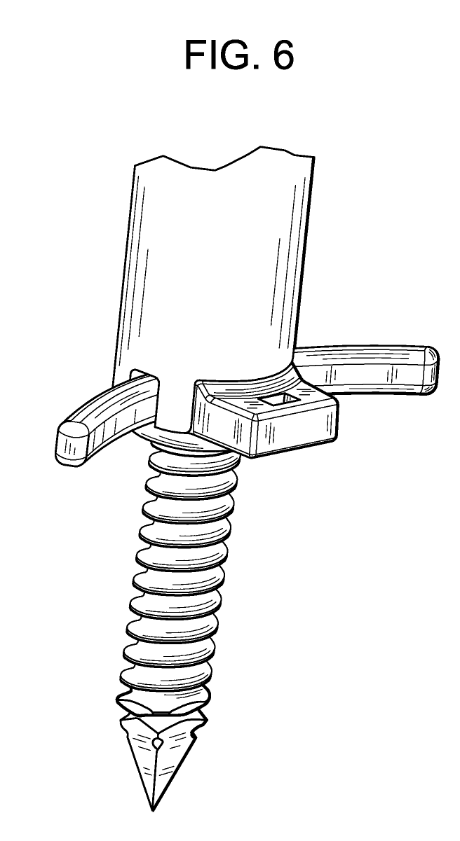

Now referring to FIG.6, the mating of the lateral edge marker features with the recesses on the bottom of the insertion tool will cause alignment of the arms of the lateral edge marker with the centering hole of the insertion tool, thereby allowing the orientation of the arms to key off the placement of the centering hole upon the midline marker.

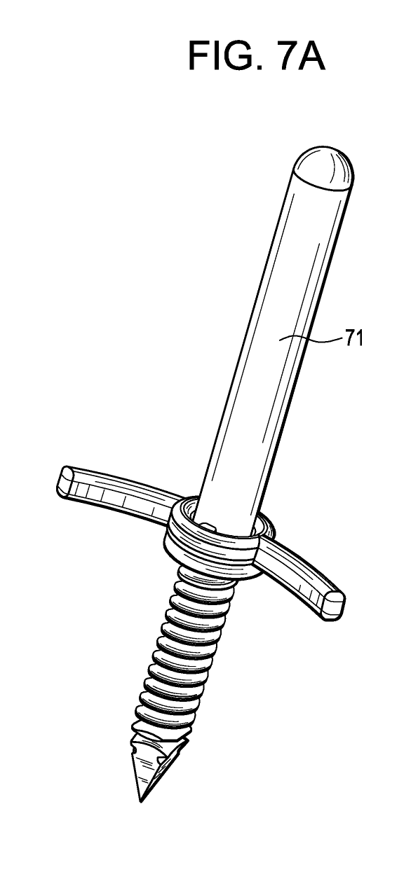

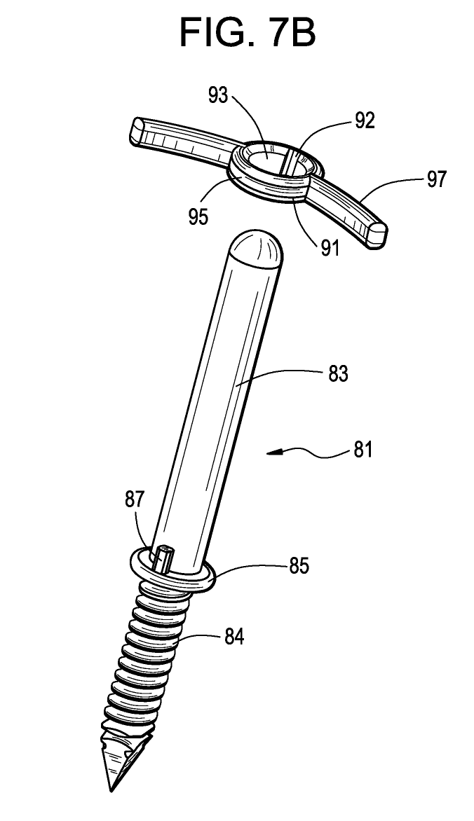

In some embodiments, and now referring to FIG. 7a, the proximal end portion 71 of the bone anchor is axially elongated so that it can function as a Caspar Pin as well as a lateral edge marker.

In some embodiments, and now referring to FIG. 7b, the arms of Caspar-type lateral edge marker are removable. The removable arms can be placed upon the casper pin at the point of care, thereby allowing modularity in the size offerings. In FIG. 7b, there is provided a modular lateral edge marker, comprising
a) a Caspar distraction pin 81 having an elongated proximal shaft 83, a distal threaded shaft 84 adapted for insertion into bone, a intermediate base 85, and tongue 87 disposed upon the proximal shaft, and
b) a ring 91 having an inner annular surface 93 having a recess 92 adapted for fixation to the tongue, an outer surface 95, and pair of arms 97 extending from the outer surface of the ring.

In other embodiments, the arms of the Caspar-type lateral edge marker are bendable to accommodate variations in size. These bendable legs are preferably made of wire and are molded to fit the specific application.

In the some embodiments of using the lateral edge marker of the present invention, the lateral edge marker pin is first inserted at the approximate midline of the vertebral body. Using fluoroscopy, verify that the outer boundaries of the lateral edge marker are consistent with the lateral edges of the disc space. Lastly, use the outer boundaries created from the lateral edge marker as guidance during the discectomy, endplate preparation and final implant placement.

In some embodiments, the lateral edge marker of the present invention is used to help the surgeon with endplate preparation prior to the insertion of the motion disc. As the step of rasping the vertebral endplates is the most imprecise step of the implantation procedure, it would be useful to develop a method of increasing the precision of this step. Keying the rasping off a properly placed midline marker is of limited utility for two reasons. First, the midline marker is often visually obscured during the rasping procedure. Second, the midline marker offers little guidance to the surgeon as to the outer boundaries of the region to be rasped. By rasping with the guidance of the lateral markers of the present invention, these drawbacks are eliminated.

Because the lateral markers of the present invention are advantageously used in the implantation of intervertebral motion disc prostheses, in some embodiments of the present invention, there is provided a kit comprising:
a) a first lateral edge marker, comprising:
   i) a bone anchor having a distal end portion adapted for fixation to a vertebral body, a proximal end and a longitudinal shaft therebetween, and
   ii) a pair of arms extending from the transversely from the bone anchor, and
b) an intervertebral motion disc.

I claim:

1. A lateral edge marker for guiding the insertion of an intervertebral motion disc, comprising:
a) a bone anchor having a distal end portion adapted for fixation to a vertebral body, a proximal end and a longitudinal shaft therebetween, and
b) a pair of arms extending transversely from the bone anchor, each arm having a lateral edge.

2. The marker of claim 1 wherein the arms form an angle of about 180 degrees.

3. The marker of claim 1 wherein the arms form an angle of between about 90 and less than 180 degrees.

4. The marker of claim 1 wherein the lateral edges are substantially parallel to each other.

5. The marker of claim 1 wherein the arms are flexible.

6. A method of inserting an intervertebral motion disc, comprising the steps of:
a) providing an upper and lower lateral edge marker, each marker comprising:
i) a bone anchor having a distal end portion adapted for fixation to a vertebral body, a proximal end and a longitudinal shaft therebetween, and
ii) a pair of arms extending from the transversely from the bone anchor, each arm having a lateral edge
b) inserting the upper lateral edge marker into an upper vertebra,
c) inserting the lower lateral edge marker into a lower vertebra, so that the four edges define a window, and
d) inserting the motion disc or an instrument through the window and into a disc space between the upper and lower vertebrae.

7. The method of claim 6 wherein a step of rasping the disc space is performed before step d).

8. The method of claim 6 wherein the arms of each marker form an angle of between about 90 and 180 degrees.

9. The method of claim 6 wherein the arms of each marker form an angle of between about 90 and less than 180 degrees.

10. The method of claim 6 wherein each marker is inserted into its vertebra so that the lateral edge of each arm is adjacent a vertebral endplate.

11. The method of claim 6 wherein the lateral edges of a marker are substantially parallel to each other.

12. The method of claim 6 wherein the lateral edges of the upper and lower markers are substantially parallel to each other.

13. The method of claim 6 wherein the bone anchor of each marker is inserted into a midline of its respective vertebra.

14. The marker of claim 1 wherein the lateral edges define a line therebetween, and wherein each lateral edge is substantially normal to the line.

15. A kit comprising:
a) a first lateral edge marker, comprising:
i) a bone anchor having a distal end portion adapted for fixation to a vertebral body, a proximal end and a longitudinal shaft therebetween, and
ii) a pair of arms extending from the transversely from the bone anchor, and
b) an intervertebral motion disc.

16. The kit of claim 11 comprising a second lateral edge marker.

17. The kit of claim 15 wherein the arms form an angle of between about 90 and 180 degrees.

18. The kit of claim 15 wherein the arms form an angle of between about 90 and less than 180 degrees.

19. The kit of claim 15 wherein the lateral edges are substantially parallel to each other.

20. The kit of claim 15 wherein the lateral edges define a line therebetween, and wherein each lateral edge is substantially normal to the line.

21. A modular lateral edge marker, comprising
a) a standard distraction pin having a shaft, and
b) a ring having a pair of arms extending therefrom,
wherein the ring is adapted to be received upon and friction fit upon the shaft of the distraction pin.
